(19) United States
(12) Patent Application Publication
(10) Pub. No.: US 2026/0354921 A1
(43) Pub. Date: Nov. 12, 2026

(54) SYSTEM AND METHOD FOR MAGNETIC MANIPULATION OF TISSUE

(71) Applicants: Arizona Board of Regents on behalf of Arizona State University, Scottsdale, AZ (US); Mayo Foundation for Medical Education and Research, Rochester, MN (US)

(72) Inventors: Hamidreza Marvi, Chandler, AZ (US); Mahdi Ilami, Tempe, AZ (US); Terry Jue, San Francisco, CA (US); Norio Fukami, Fountain Hills, AZ (US)

(21) Appl. No.: 19/134,611

(22) PCT Filed: Dec. 6, 2023
(86) PCT No.: PCT/US2023/082691

§ 371 Date: May 30, 2025

Related U.S. Application Data
(60) Provisional application No. 63/386,542, filed on Dec. 8, 2022.

(51) Int. Cl. A61B 34/00; A61B 17/00; A61B 17/02; A61B 34/37

(52) U.S. Cl. CPC A61B 34/73; A61B 17/0218; A61B 34/37; A61B 34/76; A61B 2017/00876

(57) ABSTRACT

A system and a method for manipulating tissue during a surgical procedure utilize one or more tissue anchors comprising premagnetized material affixed to tissue within an animal body. At least one magnetic field source external to the animal body is moved using at least one robotic actuator, and is used to alter position of tissue anchor(s) affixed to the tissue. A robotic actuator is controllable with a user input device with end effectors configured to provide haptic feedback to a user, with such feedback being proportional to field strength, field direction, tissue anchor strain, and/or tissue displacement. Robotic ex-situ actuation may be supplemented with in-situ actuation using a premagnetized element associated with a surgical instrument. A camera and/or optical fiber may be associated with an endoscope to enable visualization of a surgical field and/or provide signals to enable responsive control of a magnetic field source.

### STATEMENT OF RELATED APPLICATION(S)

This application claims the benefit of U.S. Provisional Patent Application No. 63/386,542 filed on Dec. 8, 2022, the disclosure of which is hereby incorporated by reference herein in its entirety.

### TECHNICAL FIELD

This disclosure relates generally to systems and methods for manipulating (e.g., moving or positioning) tissue during a surgical procedure, and relates more specifically to magnetic manipulation of tissue using one or more in situ tissue anchors.

### BACKGROUND

Various types of minimally invasive surgery involve passing an endoscope or other instrument through an incision or orifice into an animal (e.g., human) body, according to categories including endoscopy, laparoscopy, and arthroscopy. Minimally invasive surgery typically has less operative trauma, other complications, and adverse effects than a corresponding open-type surgery (involving a larger incision to permit direct viewing and manipulation of tissue by a surgeon).

Endoscopic submucosal dissection (ESD) is a widely performed endoscopic therapeutic procedure that enables en-bloc resection of larger lesions, which allows for a more precise histological evaluation and reduces recurrence rates. Colorectal precancerous lesions are mostly polyps for which curative resection could be achieved by conventional endoscopic mucosal resection (EMR) or piecemeal EMR.

One challenge during performance of endoscopic procedures is manipulating (e.g., retracting) tissue to permit a desired plane to be visualized and/or dissected. During performance of conventional open-type surgery, a surgeon can retract tissue with one hand and perform suitable operations (e.g., dissection) with another hand, but endoscopic procedures typically employ a single tubular body that is ill-suited to facilitate simultaneous tissue retraction and dissection.

Magnetic tissue anchors that permit tissue within a body to be grasped and manipulated by a manually moveable magnet, positioned external to the body, have been developed for use in endoscopic submucosal dissection. See Mortagy, M., et al., “Magnetic anchor guidance for endoscopic submucosal dissection and other endoscopic procedures,” World J. Gastroenterol. 2017 April 28; 23(16): 2883-2890 (“Mortagy et al.”). Mortagy et al. disclose that a magnetically actuated tissue anchor can provide dynamic traction independent of an endoscope, by applying an external magnetic pulling force to an internal magnetic anchor that includes a magnetic weight coupled by a connecting thread to microforceps configured to anchor tissue within a body. However, Mortagy et al. disclose that limitations of magnetic anchor guided ESD include the coupling strength of magnets that decay with distance, which may limit effectiveness of the technique through thick abdominal walls. Separately, it may be challenging for a surgeon to manipulate a magnet outside a patient's body while performing other surgical operations such as tissue dissection.

In view of the foregoing, the art continues to seek improvement in systems and methods for magnetic manipulation of tissue to enhance their utility.

### SUMMARY

Aspects of the present disclosure relate to a system and method for manipulating (e.g., moving or positioning) tissue during a surgical procedure, utilizing one or more tissue anchors comprising a premagnetized material (e.g., a permanent magnet or ferroelectric magnet) affixed to tissue within an animal body. In certain implementations, at least one magnetic field source (e.g., a permanent magnet, a ferroelectric magnet, or an electromagnet) arranged external to the animal body is moved using at least one robotic actuator, and at least one magnetic field generated by the at least one magnetic field source is used to alter position of the one or more tissue anchors affixed to the tissue. An exemplary tissue anchor may include a magnetic weight coupled with a grasping or affixing element such as microforceps, a clamp, or the like. A robot actuator may be controlled by user manipulation of a user input device, which may have one or more associated end effectors to supply haptic feedback to the user through the user input device (e.g., proportional to one or more of magnetic field strength, magnetic field direction, tissue anchor strain, and tissue displacement). A camera and/or optical fiber associated with an endoscope may be provided within the animal body in or adjacent to a surgical field (e.g., proximate to the one or more tissue anchors and/or a surgical tool) to enable visualization. In certain embodiments, movement of at least one magnetic field source may be controlled responsive to signals received from a camera within the animal body. In certain embodiments, robotic ex-situ actuation of at least one magnetic tissue anchor may be supplemented with, or replaced with, in-situ actuation using at least one premagnetized element associated with a surgical instrument, wherein the premagnetized element may be moved to alter position of one or more magnetic tissue anchors. A surgical instrument may include an elongated body structure supporting at least one premagnetized element, wherein the elongated body structure may comprise one or more of a hollow tube, a catheter, an electrical conductor, a camera, and an optical fiber. As used herein, the term “animal body” is intended to encompass a body of a human or non-human animal.

In one aspect, the disclosure relates to a method for moving or positioning tissue during a surgical procedure, the method comprising: attaching one or more tissue anchors comprising a premagnetized material to tissue within an animal body; moving at least one magnetic field source arranged external to the animal body, using at least one robotic actuator; and applying at least one magnetic field generated by the at least one magnetic field source to alter position of the one or more tissue anchors affixed to the tissue.

In certain embodiments, the at least one magnetic field source comprises a plurality of magnetic field sources.

In certain embodiments, the at least one robotic actuator comprises a plurality of robotic actuators.

In certain embodiments, the premagnetized material comprises a permanent magnet, or comprises a ferroelectric magnet.

In certain embodiments, the method further comprises controlling position of the at least one robotic actuator and magnetic field strength applied by the at least one magnetic field source by user manipulation of a user input device.

In certain embodiments, the method further comprises supplying haptic feedback to a user through the user input device proportional to at least one of the following: magnetic field strength, magnetic field direction, tissue anchor strain, and tissue displacement.

In certain embodiments, movement of the at least one magnetic field source is controlled responsive to signals received from a camera of a surgical instrument positioned within the animal tissue proximate to the at least one tissue anchor.

In certain embodiments, the method further comprises positioning a surgical instrument comprising an elongated body structure and at least one premagnetized element within the animal body proximate to the one or more tissue anchors, and moving the at least one premagnetized element of the surgical instrument within the animal body to alter position of the one or more tissue anchors affixed to the tissue.

In certain embodiments, the at least one premagnetized element comprises a permanent magnet or a ferroelectric magnet.

In certain embodiments, the at least one premagnetized element comprises an electromagnet.

In certain embodiments, the surgical instrument comprises a first channel permitting passage of a camera or an optical fiber, and comprises a second channel permitting passage of the at least one premagnetized element.

In certain embodiments, the at least one premagnetized element is rigidly coupled to the surgical instrument.

In certain embodiments, the elongated body structure of the surgical instrument comprises one or more of a hollow tube, a catheter, an electrical conductor, a camera, and an optical fiber.

In another aspect, the disclosure relates to a system configured for moving or positioning tissue during a surgical procedure, the system comprising: one or more tissue anchors comprising a premagnetized material and configured to be attached to tissue within an animal body; at least one magnetic field source configured to be arranged external to an animal body; at least one robotic actuator configured to move the at least one magnetic field source to effectuate movement of the one or more tissue anchors when attached to the tissue; and a user input device configured to receive input signals from a user to control movement of the at least one robotic actuator.

In certain embodiments, the user input device comprises at least one feedback actuator configured supplying haptic feedback to a user through the user input device, wherein the haptic feedback is proportional at least one of the following: magnetic field strength, magnetic field direction, tissue anchor strain, and tissue displacement.

In certain embodiments, the at least one magnetic field source comprises a plurality of magnetic field sources.

In certain embodiments, wherein the at least one robotic actuator comprises a plurality of robotic actuators.

In certain embodiments, the premagnetized material comprises a permanent magnet, or comprises a ferroelectric magnet.

In another aspect, the disclosure relates to a method for moving or positioning tissue during a surgical procedure, the method comprising: affixing one or more tissue anchors comprising a premagnetized material to tissue within an animal body; positioning a surgical instrument comprising an elongated body structure and at least one premagnetized element within the animal body proximate to the one or more tissue anchors; and moving the at least one premagnetized element to alter position of the one or more tissue anchors affixed to the tissue.

In certain embodiments, the at least one premagnetized element comprises a permanent magnet or a ferroelectric magnet.

In certain embodiments, the at least one premagnetized element comprises an electromagnet.

In certain embodiments, the surgical instrument comprises a first channel permitting passage of a camera or an optical fiber, and comprises a second channel permitting passage of the at least one premagnetized element.

In certain embodiments, the at least one premagnetized element is rigidly coupled to the surgical instrument.

In certain embodiments, the elongated body structure of the surgical instrument comprises one or more of a hollow tube, a catheter, an electrical conductor, a camera, and an optical fiber.

In another aspect, any two or more features of aspects and/or embodiments disclosed herein may be combined for additional advantage.

### BRIEF DESCRIPTION OF THE DRAWINGS

FIG. 1 schematically illustrates a tissue anchor arranged within a gastric cavity of an animal body with a grasping or affixing element thereof attached to a portion of gastric wall tissue, and illustrates an ex-situ robotic manipulator arranged to apply a magnetic field to a premagnetized portion of the tissue anchor.

FIG. 2 schematically illustrates interconnections between components of a system for moving or positioning tissue during a surgical procedure according to one embodiment, the system including an in-situ tissue anchor, at least one ex-situ robotic arm, a steerable surgical instrument insertable into an animal body, and various sensing and control components.

FIG. 3 is a perspective view of a robotic arm incorporating magnets to serve as an end effector to actuate one or more tissue anchors and/or effectuate movement of a steerable assembly including a magnetic needle within tissue of an animal body according to certain embodiments.

FIG. 4 schematically illustrates a portion of a fiber bragg grating (FBG) sensor that may be utilized with components for determining position of a steerable assembly (e.g., a surgical instrument) within tissue of an animal body according to certain embodiments.

FIG. 5 is a schematic diagram of a generalized representation of a computer system that can be included as one or more components of a system or method for manipulating tissue during a surgical procedure as disclosed herein.

### DETAILED DESCRIPTION

Aspects of the present disclosure relate to a system and method for manipulating (e.g., moving or positioning) tissue during a surgical procedure, utilizing one or more tissue anchors comprising a premagnetized material affixed to tissue within an animal body. An exemplary tissue anchor may include a magnetic weight coupled with a grasping or affixing element such as microforceps, a clamp, or the like. The magnetic anchor is manipulated by a magnetic field. In certain implementations, at least one magnetic field source arranged external to the animal body is moved using at least one robotic actuator, and at least one magnetic field generated by the at least one magnetic field source is used to alter position of the one or more tissue anchors affixed to the tissue. The magnetic field source external to the animal body may comprise one or more paramagnetic, ferromagnetic, and/or electromagnetic materials. A robotic actuator (e.g., an articulating robotic arm) may be moved as desired around an animal body to adjust the magnetic field strength and magnetic field direction applied to magnetic tissue anchors. Magnetic field strength and direction may be calculated based on a surgeon's desired manipulation of target tissue.

A robotic actuator may be controlled by user manipulation of a user input device, which may have one or more associated actuators to supply haptic feedback to the user through the user input device (e.g., proportional to one or more of magnetic field strength, magnetic field direction, tissue anchor strain, and tissue displacement. One example of a user input device is a joystick, which may be provided in single or dual forms, optionally augmented with various items such as triggers, buttons, dials, and the like. A camera and/or optical fiber associated with an endoscope may be provided within the animal body in or adjacent to a surgical field (e.g., proximate to the one or more tissue anchors and/or a surgical tool) to enable visualization, such as by using one or more displays, whether in stand-alone or wearable (e.g., headset) form. In certain embodiments, movement and/or activation of at least one magnetic field source may be controlled responsive to one or more of: (i) signals received from a camera within the animal body (which may detect tissue displacement), (ii) detected tissue anchor strain (such as may be detected with a strain gauge associated with the anchor), (iii)) detected magnetic field strength, and (iv) detected magnetic field direction.

In certain embodiments, robotic ex-situ actuation of at least one magnetic tissue anchor may be supplemented with, or replaced with, in-situ actuation using at least one premagnetized element associated with a surgical instrument, wherein the premagnetized element may be moved to alter position of one or more magnetic tissue anchors. A surgical instrument may include an elongated body structure supporting at least one premagnetized element, wherein the elongated body structure may comprise one or more of a hollow tube, a catheter, an electrical conductor, a camera, and an optical fiber. In certain embodiments, in-situ actuation may be used to facilitate engagement of a magnetic tissue anchor with targeted tissue, and ex-situ actuation may be used thereafter during a surgical procedure, to free up a surgical instrument for use in dissection or other operations. In certain embodiments, in-situ actuation and ex-situ actuation may be performed at different times, or may be performed simultaneously. In certain embodiments, in-situ actuation may be performed by moving a surgical instrument (e.g., a steerable surgical element) comprising at least one premagnetized element associated with an elongated structure within an animal body, wherein such actuation may be performed manually by a surgeon or aided by one or more actuators.

In certain embodiments, a surgical instrument may be steered via pushing, by exploiting asymmetric forces on an instrument (e.g., needle) tip during insertion. As the instrument tip is pushed forward through tissue, it also moves slightly sideways, motivated by the radial component of the force acting on the tip. The magnitude of this sideways movement depends on the tip geometry, tip stiffness, tissue stiffness, bevel angle, and other properties of the instrument tip-tissue interactions. The instrument (or an associated tubular structure connected to the needle) is rotated at the base to control the orientation of the tip, thus rotating the direction of the asymmetric force and permitting the trajectory of the instrument tip to be controlled.

In certain embodiments, a surgical instrument may comprise a magnetically responsive tip and by steered via magnetic pulling, by being used in conjunction with an instrument needle steering apparatus and method that alters strength and/or position of at least one magnetic field source (e.g., generated by one or more end effectors such as one or more robotic arm(s)) external to an animal body to interact with the instrument tip inserted into the animal body to effectuate movement of the instrument within the animal body. A conventional elongated structure (e.g., shaft) of the surgical instrument may be replaced by an elastic shaft that is not load-bearing. By pulling the instrument tip through tissue using externally applied magnetic forces instead of pushing at the base of a load-bearing shaft supporting a needle, any concern of shaft buckling is eliminated by avoiding formation of compression stresses in the shaft. Additional details regarding magnetic pulling of a surgical instrument through tissue are disclosed in Interntional Publication WO 2021/108690 A1, which is hereby incorporated by reference herein.

As noted previously, an exemplary tissue anchor may include a magnetic weight (comprising a premagnetized material such as a permanent magnet or a ferroelectric magnet) coupled with a grasping or affixing element such as microforceps, a clamp, or the like. The magnetic weight is tethered to the grasping or affixing element with a coupling element, which may comprise a thread and/or a flexible rod. When a magnetic field is applied to the magnetic weight (whether by a magnetic field source external to the animal body, and/or by a premagnetized element of a surgical instrument within the animal body), the magnetic weight is pulled in the direction of the applied magnetic field, thereby applying tension to the coupling element, which pulls the grasping or affixing element. Since the grasping or affixing element is coupled with tissue within the animal body, application of a magnetic field causes the tissue to be locally displaced, which may provide access and/or visibility to a surgeon to perform a desired surgical procedure.

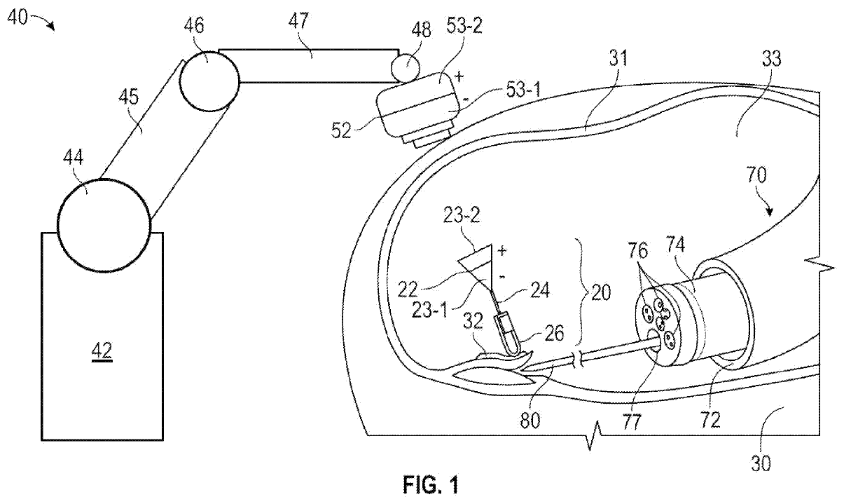

FIG. 1 schematically illustrates a tissue anchor 20 (including a magnetic weight 22 of premagnetized material, a coupling element 24, and a grasping or affixing element 26) arranged within a gastric cavity 33 of an animal body 30, with the grasping or affixing element 26 attached to a portion of gastric wall tissue 32. A robotic manipulator 40 having an associated magnetic field source 52 is positioned external to the animal body 30, wherein the magnetic field source 52 is arranged to apply a magnetic field to apply an attracting force to the magnetic weight 22, thereby applying tension to the coupling element 24 and the grasping or affixing element 26 to pull the attached portion of gastric wall tissue 32 to provide access to an implement 80 (e.g., needle, cutting instrument, etc.) of an endoscopic device 70 or other surgical instrument. As shown, the endoscopic device 70 includes a flexible body structure 74 and may include multiple bores or channels 76, 77 defined therein to receive items such as a camera, an optical fiber, and/or electrical conductors, wherein the bores or channels 76, 77 may also permit therapeutic or diagnostic material to be supplied to a surgical site, or permit tissue to be removed from a surgical site. The robotic manipulator 44 may include one or more robotic arms 45, 47 and associated joints 44, 46, 48 with multiple degrees of freedom, and/or may include multiple magnetic field sources 52. Each magnetic field source 52 includes magnets 23-1, 23-2 (e.g., permanent magnets or electromagnets), wherein in certain embodiments, the magnets 23-1, 23-2 may be, or may be controlled to be, of the same polarity or opposing polarities. Although a gastric wall 31 and gastric cavity 33 are shown, it is to be appreciated that any embodiments herein may be used with any suitable tissue within an animal (including but not limited to human) body.

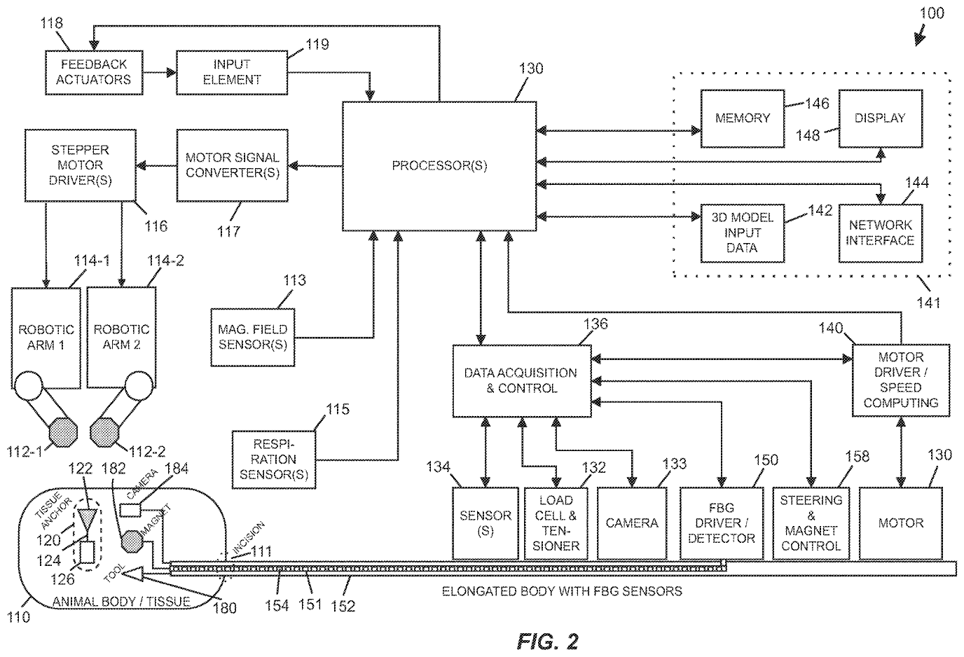

FIG. 2 schematically illustrates components of a system 100 for moving or positioning tissue during a surgical procedure according to one embodiment. At lower left, an elongated structure (e.g., surgical instrument) 152 extends through an opening or incision 111 and is positioned within tissue of an animal body 110 proximate to a tissue anchor 120 that comprises a premagnetized material 122 coupled by a coupling element 124 to a grasping or affixing element 126. The elongated structure 152 (e.g., surgical instrument) terminates at a tip 180 within the animal body 110, with the tip 180 comprising one or more of: a tool or other implement, a camera 184, and a premagnetized element 182, wherein any or all of the foregoing elements may be selectively deployed in certain embodiments. The elongated structure 152 may further comprise a plurality of fiber bragg grating (FBG) sensors 154 associated with an optical fiber 151 arranged in or on the elongated structure. Robotic manipulators 114-1, 114-2 each having an associated magnetic field source 112-1, 112-1 are positioned external to the body 110. The robotic manipulators 114-1, 114-2 may comprise robotic actuators (e.g., robotic arms, such as 6-degree-of-freedom (6 DOF) robotic arms) arranged external to the animal body 110 to apply at least one magnetic field to manipulate and cause movement of the tissue anchor 120 within the animal body 110.

The robotic manipulators 114-1, 114-2 may be controlled by stepper motor drivers 116 and a processor 130 (e.g., integrated with a microcomputer in certain embodiments), wherein one or more intermediately arranged motor signal converters 117 may also be provided. Desired poses of the robotic manipulators 114-1, 114-2 may be calculated by the processor 130 and supplied to the stepper motor drivers 116 to control movement of the robotic manipulators 114-1, 114-2. Movement of one or more magnetic end effectors 112-1, 112-2 (which may be embodied in permanent magnet materials, ferromagnetic materials, or electromagnets) may cause manipulation and/or movement of the tissue anchor 120 couplable to tissue within the animal body 110.

In certain embodiments, the tissue anchor 120 may be additionally, or alternatively, controlled by movement of the premagnetized element 182 of the elongated body 152 (e.g., surgical instrument) located within the animal body 110 proximate to the tissue anchor 120. A magnetic steering and control element 158 associated with the surgical instrument 152 may be used to control positioning (and/or applied field if the premagnetized element 182 comprises an electromagnet) of the premagnetized element 182 of the surgical instrument 152. In certain embodiments, a premagnetized implement (e.g., needle tip) 180 is associated with the elongated structure 152 of a surgical instrument and may be moved through the animal tissue 110 by magnetic pulling with the robotic manipulators 114-1, 114-2 and magnetic effectors 112-1, 112-2 located external to the animal body, and after the premagnetized implement 180 is positioned in a surgical field, the premagnetized implement 180 may be retracted through a bore or channel (e.g., 76, 77 in FIG. 1) of the elongated structure 152, and a magnetically moveable tissue anchor 120 may be deployed through a bore or channel (e.g., 76, 77 in FIG. 1) of the elongated structure 152 into a surgical field within the animal tissue 110.

A user input device 119 controllable by user manipulation is arranged to permit control of the magnetic end effectors 112-1, 112-2. One or more feedback actuators 118 may be configured to supply haptic feedback to the user through the user input device 119 (e.g., proportional to one or more of magnetic field strength, magnetic field direction, tissue displacement, tissue density, deviation from desired trajectory, or the like). One example of a user input device 119 is a joystick, which may be provided in single or dual forms, optionally augmented with various items such as triggers, buttons, dials, and the like. A camera and/or optical fiber (coupled to camera imager 133) associated with the elongated body structure 152 may be provided within the body 110 (optionally within a surgical field for an animal body 110, such as proximate to a surgical tool at a top 180 of the elongated body 152) to enable visualization, such as by using one or more displays 148, whether in stand-alone or wearable (e.g., headset) form.

In certain embodiments, movement and/or activation of at least one magnetic field source 112-1, 112-2 may be controlled responsive to one or more of: (i) signals received from a camera 184 within the animal body (which may detect tissue displacement), (ii) detected tissue anchor strain (such as may be detected with a strain gauge associated with the anchor 120), (iii)) detected magnetic field strength, and (iv) detected magnetic field direction. Magnetic field strength and/or field direction may be detected by one or more magnetic field sensors 113 arranged external to the animal body 110.

In certain embodiments, position of the surgical instrument within the animal body may be estimated without continuous imaging techniques. In certain embodiments, one or more fiber bragg grating (FBG) sensors 154 may be provided on an optical fiber 151 arranged in or on the elongated body structure 152 of a surgical instrument and inserted into tissue of an animal body 110 to determine position of the elongated body structure. Light signals may be supplied to FBG sensors by a FBG driver/detector 150 arranged external to the animal body 110. Reflected light signals received by the FBG driver/detector 150 may be used to determine one or more of force, strain, or shape of FBG sensors 150 associated with the elongated body structure 152, and thereby used to determine orientation of the elongated body structure 152.

In certain embodiments, a tracking subsystem for the elongated body 152 may include a DC motor 130 having a rotatable spool coupled thereto, a load cell and tensioner 132, and a rotary encoder (optionally integrated into a motor driver/speed computing element 140 coupled to the DC motor 130). The foregoing items may be mounted on a moveable support structure (not shown), such as a platform mounted on linear guides that enable one-directional (e.g., horizontal) translation in one direction. One example of a moveable support structure that may be used is shown in International Publication No. WO 2021/108690 A1, with the disclosure thereof being hereby incorporated by reference herein. A portion of the elongated body structure 152 may be wrapped on the spool coupled with a shaft of the DC motor 130. The load cell 132 (or alternatively a force sensor) may be used to measure tensile or compressive loads applied to the moveable support structure, wherein the processor 130 may be used in combination with the rotary encoder (e.g., within motor driver/speed computing element 140) to calculate rotational velocity of the motor 130, which may be used to calculate insertion depth of the elongated body 152 in the body 110. Measurements from the load cell 132 may be used to calculate tension applied to the elongated structure 152. A data acquisition device 136 sends control inputs to a motor driver 140 that supplies power to the DC motor 130 to which the elongated structure 152 is coupled.

In certain embodiments, the DC motor 130 may be used to provide controlled releasement of the elongated body structure 152 from a spool of the motor 130. The processor 130 may be used to compare an output signal of at least one sensor 134 configured to sense a condition indicative of at least one of (i) position of a moveable support structure or (ii) pulling force applied to a moveable support structure, and configured to generate at least one output signal. In certain embodiments, operation of the DC motor 130 may be controlled to adjust a feed rate of a length of elongated body structure 152 from a rotatable spool of the motor 130 responsive to comparison of the output signal to the desired range of output signal values. For example, if tension on the elongated body structure 152 is too high, then in certain embodiments, operation of the DC motor 130 may be controlled to increase the releasement rate of the elongated body structure 152 from the rotatable spool of the motor. In certain embodiments, operation of the DC motor 130 may be controlled to reverse rotational direction of the motor 130 responsive to comparison of the output signal to the desired range of output signal values

In certain embodiments, a three-dimensional (3D) model of tissue of an animal body is generated before a steerable assembly including the elongated body structure 152 (e.g., surgical instrument) is supplied to tissue of the animal body 110. Such a 3D model may be generated by any suitable imaging device, such as a MRI, CT, ultrasound, fluoroscopy, or other imaging device. The 3D model, optionally received via a network interface 144 and/or generated from 3D model input data 142 as part of a 3D model interaction subsystem 141, may be stored to memory 146 accessible to at least one processor 130, in preparation for receiving 3D trajectory information of a steerable assembly (including the elongated body 152) for superimposition onto the 3D model. This 3D trajectory information may be determined by directly by imaging, or inferentially from a detected length of insertion of the elongated structure 152 into the animal body 110, in combination with a recorded directionality of a magnetic field applied (by magnetic effectors 112-1, 112-2) to a premagnetized material (e.g., magnetic tip 180) associated with the elongated body 152, optionally embodied in a surgical instrument.

In certain embodiments, insertion length of the elongated body structure 152 may be determined (or supplemented) by sensing position or velocity of a shaft of the DC motor 130 controlling releasement of the elongated body structure 152 during insertion of the elongated body structure 152 into the animal body 110. In certain embodiments, position or velocity of a shaft of the motor 130 may be sensed with a rotary encoder, which may be integrated into a motor driver/speed computing element 140. In certain embodiments, insertion length of the elongated body structure 152 may be determined by sensing linear position or displacement of at least a portion of the elongated body structure 152, such as by using a linear encoder (not shown) arranged between a spool coupled to the motor 130 and the animal body 110.

In certain embodiments, recording of directionality of a magnetic field applied to the elongated body 152 in the animal body 110 comprises recording control signals supplied to the stepper motor drivers 116 coupled with the robotic manipulators 114-1, 114-2 configured to adjust position of magnetic end effectors 112-1, 112-1 configured to apply one or more magnetic fields to a tip 180 of the elongated body 152. In certain embodiments, recording of directionality of the magnetic field may comprise, or be supplemented by, collecting signals received from one or more magnetic field sensors 113. In certain embodiments, one or more magnetic field sensors 113 may be positioned proximate to the animal body 110 into which the elongated body 152 is inserted.

In certain embodiments, a condition indicative of respiration rate and/or respiration amplitude of an animal body 110 may be sensed (e.g., using respiration sensors 115 and/or a ventilator or one or more chest sensors), and responsive to the such sensing, a 3D model of the animal body 110 (storable in memory 146) may be updated, and/or position of the magnetic end effectors 114-1, 114-2 may be adjusted. For an animal body 110 arranged in a lying position, the foregoing control scheme may be used to maintain constant distance in the vertical direction between the tissue of the animal body 110 and the magnetic end effectors 114-1, 114-2 so that a constant magnetic force is applied on a premagnetized needle at a tip 180 of the elongated body 152.

While continuous imaging of an animal body 110 is not required according to methods disclosed herein, in certain embodiments a body imaging apparatus (not shown) arranged external to the animal body 110 may be provided to periodically permit imaging of the body 110 and inserted portions of the elongated structure 152, as may be useful to confirm and/or correct FBG-calculated positional information derived from the FBG sensors 154 and FBG detector 150.

In certain embodiments, the system 100 may be configured to receiving signals for linear translation of an elongated body (for determining insertion depth of the elongated body structure 152) and signals for movement of the robotic manipulators 114-1, 114-2 (for determining magnetic field direction) and processing the signals for forwarding to a computer processor 130 for superimposition of 3D trajectory of the elongated body 152 (e.g., optionally embodied in a surgical instrument) on a previously generated 3D model of tissue of an animal body 110 into which the elongated body 152 inserted.

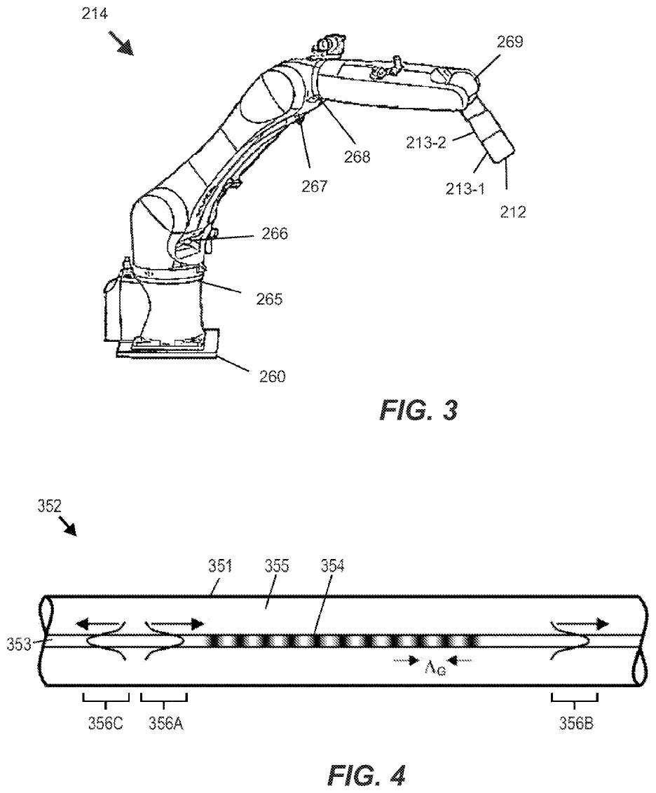

FIG. 3 is a perspective view of a robotic arm 214 incorporating magnets 213-1, 213-2 (e.g., permanent magnets or electromagnets) to serve as an end effector 212 to effectuate movement of a steerable assembly including a magnetic needle within tissue of an animal body according to certain embodiments. In certain embodiments, the magnets 213-1, 213-2 may be, or may be controlled to be, of the same polarity or opposing polarities. The robotic arm 214 is mountable to a support surface 260 and includes multiple joints 265-269 to provide numerous degrees of freedom for movement of the robotic arm 214 relative to tissue of an animal body (not shown) in order to effectuate movement of a tissue anchor (e.g., 120 in FIG. 2) within an animal body, or to effectuate movement of an implement including a premagnetized portion (e.g., needle tip 180 in FIG. 2) of a surgical instrument within tissue of the animal body. In certain embodiments, the robotic arm 213 may be used initially to move an implement within tissue of the animal body, and thereafter to manipulate a tissue anchor.

FIG. 4 is a schematic view illustration of a portion of a fiber bragg grating (FBG) sensor 352 that may be utilized with components for determining position of a steerable assembly (e.g., a surgical instrument) within tissue of an animal body according to certain embodiments. The FBG sensor 352 is embodied in an optical fiber 351 having a core 353 surrounded by cladding 355. A portion of the core 353 constitutes an index modulation region 354 in which an index of refraction of glass material of the core 353 periodically varies. When an input signal 356A (having a propagating core mode) is transmitted through the core 353 and reaches the index modulation region 354, one spectral portion of the input signal is reflected to produce a reflected signal 356C, while another spectral portion is transmitted through the index modulation region 354 to provide a transmitted signal 356B. The reflected signal 356C may be detected by a light detector associated with a FBG driver/detector unit (not shown), and analyzed to determine one or more of force, strain, or shape experienced by the FBG sensor 352. In certain embodiments, one or more FBG sensors may be arranged in or on an elongated body structure of a steerable assembly, wherein an index modulation region may be provided proximate to a magnetic needle affixed to the elongated structure.

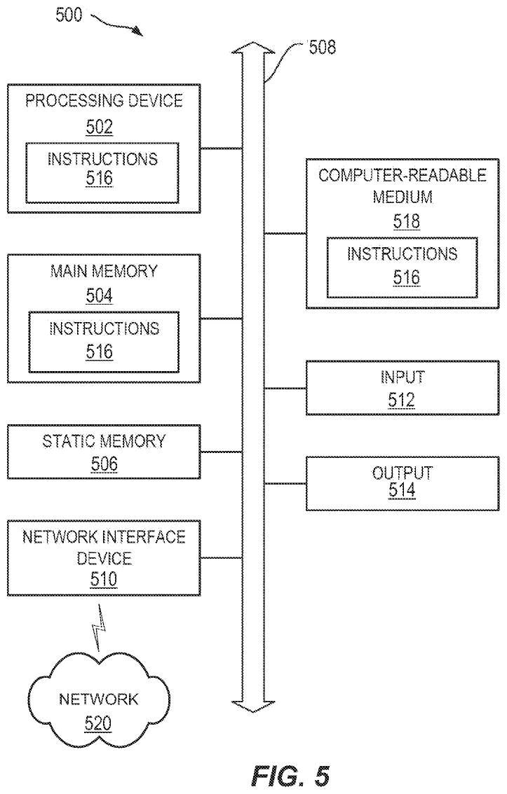

FIG. 5 is a schematic diagram of a generalized representation of a computer system 500 that can be included as one or more components of a system or method for manipulating tissue (optionally in conjunction with steering a magnetic implement) during a surgical procedure as disclosed herein, according to one embodiment. The computer system 500 may be adapted to execute instructions from a computer-readable medium to perform these and/or any of the functions or processing described herein.

The computer system 500 may include a set of instructions that may be executed to program and configure programmable digital signal processing circuits for supporting scaling of supported communications services. The computer system 500 may be connected (e.g., networked) to other machines in a local area network (LAN), an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 500 may be a circuit or circuits included in an electronic board or card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 500 in this embodiment includes a processing device or processor 502, a main memory 504 (e.g., read-only memory (ROM), flash memory, dynamic random access memory (DRAM), such as synchronous DRAM (SDRAM), etc.), and a static memory 506 (e.g., flash memory, static random access memory (SRAM), etc.), which may communicate with each other via a data bus 508. Alternatively, the processing device 502 may be connected to the main memory 504 and/or static memory 506 directly or via some other connectivity means. The processing device 502 may be a controller, and the main memory 504 or static memory 506 may be any type of memory.

The processing device 502 represents one or more general-purpose processing devices, such as a microprocessor, central processing unit (CPU), or the like. In certain embodiments, the processing device 502 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 502 is configured to execute processing logic in instructions for performing the operations and steps discussed herein.

The computer system 500 may further include a network interface device 510. The computer system 500 may additionally include at least one input 512, configured to receive input and selections to be communicated to the computer system 500 when executing instructions. The computer system 500 also may include an output 514, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 500 may or may not include a data storage device that includes instructions 516 stored in a computer readable medium 518. The instructions 516 may also reside, completely or at least partially, within the main memory 504 and/or within the processing device 502 during execution thereof by the computer system 500, the main memory 504 and the processing device 502 also constituting computer readable medium. The instructions 516 may further be transmitted or received over a network 520 via the network interface device 510.

While the computer readable medium 518 is shown in an embodiment to be a single medium, the term “computer-readable medium” should be taken to include a single medium or multiple media (e.g., a centralized or distributed database, and/or associated caches and servers) that store the one or more sets of instructions. The term “computer readable medium” shall also be taken to include any medium that is capable of storing, encoding, or carrying a set of instructions for execution by the processing device and that cause the processing device to perform any one or more of the methodologies of the embodiments disclosed herein. The term “computer readable medium” shall accordingly be taken to include, but not be limited to, solid-state memories, an optical medium, and/or a magnetic medium.

Those skilled in the art will recognize improvements and modifications to the preferred embodiments of the present disclosure. All such improvements and modifications are considered within the scope of the concepts disclosed herein and the claims that follow. Any of the various features and elements as disclosed herein may be combined with one or more other disclosed features and elements unless indicated to the contrary herein.

1. A method for moving or positioning tissue during a surgical procedure, the method comprising:
attaching one or more tissue anchors comprising a premagnetized material to tissue within an animal body;
moving at least one magnetic field source arranged external to the animal body, using at least one robotic actuator;
applying at least one magnetic field generated by the at least one magnetic field source to alter position of the one or more tissue anchors affixed to the tissue;
controlling (i) position of the at least one robotic actuator and (ii) magnetic field strength applied by the at least one magnetic field source, by user manipulation of a user input device; and
supplying haptic feedback to a user through the user input device proportional at least one of the following: magnetic field strength, magnetic field direction, tissue anchor strain, and tissue displacement.

2. The method of claim 1, wherein the at least one magnetic field source comprises a plurality of magnetic field sources, and/or the at least one robotic actuator comprises a plurality of robotic actuators.

3. The method of claim 1, wherein the at least one magnetic field source comprises one or more of an electromagnet, a permanent magnet, and a ferroelectric magnet.

4. (canceled)

5. (canceled)

6. (canceled)

7. (canceled)

8. The method of claim 1, wherein movement of the at least one magnetic field source is controlled responsive to signals received from a camera of a surgical instrument positioned within the animal tissue proximate to the at least one tissue anchor.

9. The method of claim 1, further comprising positioning a surgical instrument comprising an elongated body structure and at least one premagnetized element within the animal body proximate to the one or more tissue anchors, and moving the at least one premagnetized element of the surgical instrument to alter position of the one or more tissue anchors affixed to the tissue.

10. The method of claim 9, wherein the at least one premagnetized element of the surgical instrument comprises a permanent magnet or a ferroelectric magnet.

11. The method of claim 9, wherein the at least one premagnetized element of the surgical instrument comprises an electromagnet.

12. The method of claim 9, wherein the surgical instrument comprises a first channel permitting passage of a camera or an optical fiber, and comprises a second channel permitting passage of the at least one premagnetized element.

13. The method of claim 9, wherein the at least one premagnetized element is rigidly coupled to the surgical instrument.

14. The method of claim 9, wherein the elongated body structure comprises a hollow tube or a catheter.

15. (canceled)

16. (canceled)

17. The method of claim 9, wherein the elongated body structure contains at least one of a camera, an optical fiber, and an electrical conductor.

18. A system configured for moving or positioning tissue during a surgical procedure, the system comprising:
one or more tissue anchors comprising a premagnetized material and configured to be attached to tissue within an animal body;
at least one magnetic field source configured to be arranged external to the animal body,
at least one robotic actuator configured to move the at least one magnetic field source to effectuate movement of the one or more tissue anchors when attached to the tissue; and
a user input device configured to receive input signals from a user to control movement of the at least one robotic actuator, wherein the user input device comprises at least one feedback actuator configured supplying haptic feedback to a user through the user input device, wherein the haptic feedback is proportional at least one of the following: magnetic field strength, magnetic field direction, tissue anchor strain, and tissue displacement.

19. (canceled)

20. The system of claim 18, wherein the at least one magnetic field source comprises a plurality of magnetic field sources.

21. The system of claim 18, wherein the at least one robotic actuator comprises a plurality of robotic actuators.

22. (canceled)

23. A method for moving or positioning tissue during a surgical procedure, the method comprising:
affixing one or more tissue anchors comprising a premagnetized material to tissue within an animal body;
positioning a surgical instrument comprising an elongated body structure and at least one premagnetized element within the animal body proximate to the one or more tissue anchors; and
moving the at least one premagnetized element to alter position of the one or more tissue anchors affixed to the tissue.

24. The method of claim 23, wherein the at least one premagnetized element comprises a permanent magnet or a ferroelectric magnet.

25. The method of claim 23, wherein the at least one premagnetized element comprises an electromagnet.

26. The method of claim 23, wherein the surgical instrument comprises a first channel permitting passage of a camera or an optical fiber, and comprises a second channel permitting passage of the at least one premagnetized element.

27. The method of claim 23, wherein the at least one premagnetized element is rigidly coupled to the surgical instrument.

28. The method of claim 23, wherein the elongated body structure of the surgical instrument comprises one or more of a hollow tube, a catheter, an electrical conductor, a camera, and an optical fiber.
